SEQUENCE LISTING (13 sequences)

SEQ ID NO: 1 - protein, 7 aa, Artificial Sequence
  Synthetic
SSSYYWG

SEQ ID NO: 2 - protein, 16 aa, Artificial Sequence
  Synthetic
SFFYTGSTYYNPSLRS

SEQ ID NO: 3 - protein, 17 aa, Artificial Sequence
  Synthetic
QSTYYYGSGNYYGWFDR

SEQ ID NO: 4 - protein, 11 aa, Artificial Sequence
  Synthetic
RASQSVSSYLA

SEQ ID NO: 5 - protein, 7 aa, Artificial Sequence
  Synthetic
DASNRAT

SEQ ID NO: 6 - protein, 9 aa, Artificial Sequence
  Synthetic
QQRSNWPPA

SEQ ID NO: 7 - protein, 127 aa, Artificial Sequence
  Synthetic
QLQLQESGPGLVKPSETLSLTCTVSGGSINSSSYYWGWLRQSPGKGLEWIGSFFYTGSTYYNPSLRSRLTISVDTSKNQFSLMLSSVTAADTAVYYCARQSTYYYGSGNYYGWFDRWDQGTLVTVSS

SEQ ID NO: 8 - protein, 107 aa, Artificial Sequence
  Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGQGTKVEIK

SEQ ID NO: 9 - protein, 476 aa, Artificial Sequence
  Synthetic
MGWSCIILFLVATATGVHSQLQLQESGPGLVKPSETLSLTCTVSGGSINSSSYYWGWLRQSPGKGLEWIGSFFYTGSTYYNPSLRSRLTISVDTSKNQFSLMLSSVTAADTAVYYCARQSTYYYGSGNYYGWFDRWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 233 aa, Artificial Sequence
  Synthetic
MGWSCIILFLVATATGVHSEIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1431 bp, Artificial Sequence
  Synthetic
atgggatggtcatgtatcatcctttttctagtagcaactgcaactggagtacattcacagctgcagctgcaggagtcgggcccaggactggtgaagccttcggagaccctgtccctcacctgcactgtctctggtggctccatcaacagtagtagttactactggggctggctccgccagtccccagggaaggggctggagtggattgggagtttcttttatactgggagcacctactacaacccgtccctcaggagtcgactcaccatatccgtagacacgtccaagaaccagttctccctgatgctgagttctgtgaccgccgcagacacggctgtatattactgtgcgagacagtccacgtattactatggttcggggaattattatggctggttcgaccgctgggaccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 12 - DNA, 702 bp, Artificial Sequence
  Synthetic
atgggatggtcatgtatcatcctttttctagtagcaactgcaactggagtacattcagaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 13 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

(19) United States
(12) Patent Application Publication
(10) Pub. No.: US 2017/0137523 A1
(43) Pub. Date: May 18, 2017

(54) THERAPY FOR GIST

(71) Applicant: ImClone LLC, Indianapolis, IN (US)

(72) Inventor: Gaurav D. SHAH, New York, NY (US)

(73) Assignee: ImClone LLC, Indianapolis, IN (US)

(21) Appl. No.: 15/318,376

(22) PCT Filed: Jun. 26, 2015
(86) PCT No.: PCT/US2015/037970

§ 371 Date: Dec. 13, 2016

Related U.S. Application Data
(60) Provisional application No. 62/020,429, filed on Jul. 3, 2014.

(51) Int. Cl. C07K 16/28; C12Q 1/68

(52) U.S. Cl. CPC C07K 16/2863; C12Q 1/6886; C12Q 2600/156; C12Q 2600/158; C12Q 2600/106; A61K 2039/505

(57) ABSTRACT

The invention provides for the human antibodies that bind to human platelet-derived growth factor receptor alpha (PDGFR alpha), preferably olaratumab, for the treatment of gastrointestinal stromal tumors with PDGFR alpha mutations including D842V.

This application claims the benefit of U.S. Provisional Application No. 62/020,429 which was filed 3 Jul. 2014.

This invention is directed to the fields of immunology and cancer treatment. More specifically, the present invention is directed to olaratumab to treat gastrointestinal stromal tumors (GIST), preferably those harboring the platelet-derived growth factor receptor alpha (PDGFRα) D842V mutation, and as a medicament for the treatment of GIST.

GIST are mesenchymal neoplasms that arise predominantly in the gastrointestinal tract (GI) including the stomach and small intestine. GIST includes tumors once diagnosed (prior to the molecular profiling of GIST) as gastrointestinal leiomyomas, leiomyoblastomas, leiomyosarcomas, neurofibromas, or schwannomas. Most GIST are driven by activating mutations in the KIT tyrosine kinase receptor however, a small proportion (5%-7%) of GIST have activating mutations in the related kinase PDGFRα. Biron T. et. al. J Clin Oncol. 2010; 28:15s (suppl; abstr 10051). The KIT and PDGFRα activating mutations are mutually exclusive (Corless C., et. al. Nature Reviews, Cancer. 2011; 11: 865-878), although recent evidence indicates that drug-resistant GIST bearing KIT mutations may acquire secondary mutations to PDGFRα (Debiec-Rychter M. et. al. Gastroenterology 2005; 128:270-279). The present invention is a response to a clinically unmet need for treatment of GIST, specifically those with a PDGFRα mutation. Olaratumab for the treatment of GIST provides unexpected clinical benefit for this patient population with such mutations.

Surgical resection is the optimal approach to primary GIST without evidence of metastases. However, recurrence within five years is fairly common even for completely resected tumors. Conventional cytotoxic chemotherapy is associated with typical response rates of 5% or less, and the median survival for patients with metastatic/unresectable GIST is only 5-12 months.

Approved non-surgical treatment options include three small molecule tyrosine kinase inhibitors (TKI): imatinib (targeting KIT and PDGFRα; approved for KIT positive unresctable and/or metastatic malignant GIST), sunitinib (targeting KIT and PDGFRα; approved for GIST after disease progression on, or intolerance to, imatinib), and regorafenib (targeting KIT, PDGFRα and vascular endothelial growth factor receptor (VEGFR); approved for advanced GIST that cannot be surgically removed and no longer respond to imatinib and sunitinib), that are approved as single-agent therapy for the treatment of unresectable or metastatic GIST in the first-, second-, and third-line respectively. Corless C., et. al. Nature Reviews, Cancer. 2011; 11: 865-878.

Aberrant activation resulting in constitutive, ligand-independent activity of PDGFRα through mutation may be linked to resistance to other GIST treatments. A number of PDGFRα mutations are known (Corless C L, et. al. J Clin Oncol 2005; 23:5357-5364) however, their roles in GIST are still being be clarified. One mutation that may play a role in this resistance mechanism is PDGFRα D842V. However, the role and efficacy of targeting PDGFRα mutations, including the D842V PDGFRα mutation, are uncertain and remain the subject of investigation and debate amongst those of skill in the art. Zoler M, Published on Mar. 3, 2014 at http://www.oncologypractice.com/topics/sarcoma-gist/single-article-page/role-for-gist-genotyping-stirs-controversy/800d33412028870aef488cd0df0ca190?email=MARCHESANI@LILLY.COM&ocid=1133957.html. Accordingly, the effect and magnitude of efficacy of PDGFRα inhibitors for GIST patients with PDGFRα mutations are currently being debated.

Current data suggest that unresectable or metastatic GIST patients who have non-acquired PDGFRα-activating mutations such as PDGFRα D842V, tend to be resistant or unresponsive to imatinib (Cassier P., et. al. Clin Cancer Res. 2012; first published online at Jun. 20, 2012 and Corless C L, et. al. J Clin Oncol 2005; 23:5357-5364) and sunitinib (Heinrich M C, et. al. J Clin Oncol. 2008; 26(33):5352-5359). The median progression-free survival (PFS) of patients with PDGFRα D842V is only approximately 12 weeks despite treatment with imatinib. Biron T. et. al. J Clin Oncol. 2010; 28:15s (suppl; abstr 10051). Given the limited efficacy of imatinib and sunitinib for GIST that harbor PDGFRα mutations and for patients whose disease becomes refractory to both imatinib and sunitinib, new treatments for these subpopulations of patients are needed.

It remains unclear whether a PDGFRα inhibitor will be an effective treatment for this patient population. Recent early studies with crenolanib, a TKI small molecule inhibitor that targets PDGFRα including the D842V mutation, has shown efficacy in pre-clinical models; crenolanib is currently being investigated in a phase 2 study. Heinrich M C, et. al. Clin Cancer Res. 2012; 18; 4375. However, as a small molecule TKI, it lacks the specificity as well as potentially some of the functionality of the presently described invention. Accordingly, new, efficacious, and well-tolerated treatments for GIST that provide a clinical benefit are greatly needed for this patient population.

In short, there is a high unmet clinical need for new, efficacious, and well tolerated treatments for GIST that provide a clinical benefit. Additionally, there is a high unmet clinical need for new, efficacious, and well tolerated treatments for GIST patients whose disease becomes refractory to both imatinib and sunitinib, that provide a survival benefit. Additionally, there is a high unmet clinical need for new, efficacious, and well tolerated treatments for GIST patients with PDGFRα mutations that provide a survival benefit. More specifically, there is a high unmet clinical need for new, efficacious, and well tolerated treatments for GIST patients with the D842V PDGFRα mutation that provide a survival benefit.

A novel use of olaratumab for the treatment of GIST, especially GIST with PDGFRα mutations, more specifically the D842V mutation, is herein presented. Olaratumab, IMC-3G3 (U.S. Pat. Nos. 8,128,929 and 8,574,578), is a recombinant human monoclonal antibody which specifically targets PDGFRα. The patents disclose the treatment of a variety of neoplastic diseases, including soft tissue sarcomas, with PDGFRα antibodies, including IMC-3G3. The present invention was studied in a Phase 2 trial (http://www.clinicaltrials.gov/ct2/show/NCT01316263?term=3G3&rank=8) (hereinafter “Study”). Neither the patents nor the Study design provide any suggestion of the role or efficacy of targeting PDGFRα mutations and more specifically the D842V mutation.

The results of the Study illustrate an unexpected benefit especially for GIST patients harboring PDGFRα mutations (including D842V) as compared to PDGFRα wild-type GIST patients. The median PFS in the Study was 32.1 weeks for the PDGFRα mutated cohort as compared to 6.1 weeks for the PDGFRα wild-type/non-mutated cohort.

Furthermore, a median PFS in the Study for the PDGFRα mutated cohort of 32.1 weeks is a significant improvement over the approximately 12 weeks median PFS for PDGFRα D842V mutated patients treated with imatinib as disclosed in the art. Biron T. et. al. J Clin Oncol. 2010; 28:15s (suppl; abstr 10051).

Additionally, a clear trend can be seen in clinical efficacy: at 32 weeks, 29% of patients in the Study harboring a PDGFRα mutation had yet to demonstrate disease progression while all of the patients identified as PDGFRα wild-type, including patients with non-mutated PDGFRα, demonstrated disease progression at 32 weeks. This trend is clinically significant in light of the nature of GIST. Therefore, patients harboring a PDGFRα mutation treated with olaratumab had a clinical benefit as compared to PDGFRα wild-type patients when treated with olaratumab.

According to the first aspect of the present invention, there is provided a method of treating a gastrointestinal stromal tumor in a patient, comprising administering a therapeutically effective amount of olaratumab to the patient in need thereof, provided that a sample taken from the patient contains a PDGFR alpha mutation.

In another aspect of the invention, there is provided a method of treating a gastrointestinal stromal tumor in a patient, comprising administering a therapeutically effective amount of olaratumab to the patient in need thereof, provided that the patient is selected for treatment on the basis of a sample taken from the patient that contains a PDGFR alpha mutation.

Yet another aspect of the present invention is a method of treating a gastrointestinal stromal tumor in a patient, comprising assaying a sample taken from the patient for a PDGFR alpha mutation prior to administering olaratumab, and administering to the patient a therapeutically effective amount of olaratumab if the PDGFR alpha mutation is present in the sample.

Another aspect of the present invention is an in vitro method of selecting a patient having a gastrointestinal stromal tumor for treatment with a therapeutically effective amount of olaratumab, comprising assaying for the presence of a PDGFR alpha mutation in a sample taken from the patient, wherein the patient is selected for treatment with olaratumab if the PDGFR alpha mutation is present in the sample.

One aspect of the invention is a method of identifying a gastrointestinal stromal tumor patient eligible for treatment with olaratumab, comprising assaying for the presence of a PDGFR alpha mutation by DNA or RNA sequencing of a sample taken from the patient prior to the administration of a therapeutically effective amount of olaratumab, wherein the patient is eligible for treatment with olaratumab if the PDGFR alpha mutation is present in the sample.

Another aspect of the invention is an improved method of treating a patient having a gastrointestinal stromal tumor with olaratumab, the method comprising determining the presence of a PDGFR alpha mutation in a sample taken from the patient, and wherein the mutation is determined prior to administration of a therapeutically effective amount of olaratumab.

In a preferred aspect of the invention relating to the methods disclosed above, the olaratumab is administered at a dose of about 20 mg/kg.

One aspect of the present invention is a method of predicting the response of a gastrointestinal stromal tumor patient to treatment with olaratumab, comprising assaying a sample taken from the patient to determine the presence of a PDGFR alpha mutation in the sample, wherein the presence of a mutation in the sample is predictive of the patient's effective response to olaratumab.

Another aspect of the present invention is an in vitro method of predicting the response of a gastrointestinal stromal tumor patient to the administration of olaratumab, comprising performing DNA or RNA sequencing on a sample taken from the patient, wherein the presence of a PDGFR alpha mutation indicates an increased likelihood that the patient will effectively respond to the administration of olaratumab.

In a preferred aspect of the invention relating to the methods disclosed above, the PDGFR alpha mutation is D842V.

In a preferred aspect of the invention relating to the methods disclosed above, the sample is selected from the group consisting of blood, serum, plasma, urine, tissue, tumor cells, tumor tissue samples, circulating tumor cells, and circulating DNA.

One aspect of the invention is a therapeutic regimen for treating a gastrointestinal stromal tumor, comprising: (1) selecting a patient having a gastrointestinal stromal tumor on the basis of a sample taken from the patient having a PDGFR alpha mutation, wherein the sample is selected from the group consisting of blood, serum, plasma, urine, tissue, tumor cells, tumor tissue samples, circulating tumor cells, and circulating DNA, and (2) administering to the patient olaratumab if the mutation is present. In a preferred aspect of this invention, the mutation of PDGFR alpha is D842V. In another preferred aspect of this invention, the olaratumab is administered at a dose of about 20 mg/kg.

In another aspect of the invention, there is provided a pharmaceutical composition comprising olaratumab with one or more pharmaceutically acceptable carriers, diluents, or excipients, for use in the treatment of a gastrointestinal stromal tumor having a PDGFR alpha mutation. In a preferred aspect of this invention, the mutation of PDGFR alpha is D842V. In another preferred aspect of this invention, the olaratumab is administered at a dose of about 20 mg/kg.

Use of olaratumab in the manufacture of a medicament for the treatment of a gastrointestinal stromal tumor with a PDGFR alpha mutation is another aspect of the present invention. In a preferred aspect of this invention, the mutation of PDGFR alpha is D842V. In another preferred aspect of this invention, the olaratumab is administered at a dose of about 20 mg/kg.

One aspect of the present invention is olaratumab for use in the treatment of a gastrointestinal stromal tumor with a PDGFR alpha mutation. In a preferred aspect of this invention, the mutation of PDGFR alpha is D842V. In another preferred aspect of this invention, the olaratumab is administered at a dose of about 20 mg/kg.

Yet another aspect of the present invention is olaratumab for use in treating a gastrointestinal stromal tumor, comprising the steps: (1) assaying a sample from a patient, wherein the sample is selected from the group consisting of blood, serum, plasma, urine, tissue, tumor cells, tumor tissue samples, circulating tumor cells, and circulating DNA, (2) determining the presence of a PDGFR alpha mutation in the sample taken from the patient, wherein the mutation of PDGFR alpha is D842V, and (3) administering olaratumab to the patient if the mutation is present. In a preferred aspect of this invention, the olaratumab is administered at a dose of about 20 mg/kg.

The present invention also contemplates the following non-limiting list of embodiments, which are further described elsewhere herein:

According to a preferred embodiment of the present invention, there is provided a pharmaceutical composition of olaratumab for use in therapy of GIST wherein the olaratumab is administered on a 14-day cycle, wherein each dose of olaratumab falls within the range of about 10 mg/kg to about 30 mg/kg. Preferably the dose is in the range of about 18.5 mg/kg to about 22.5 mg/kg and most preferably is about 20 mg/kg. Preferably, patients should be treated in cycles of 14 days until evidence of confirmed disease progression.

The invention provides for olaratumab in various aspects disclosed herein. Olaratumab is an antibody specific for human PDGFR alpha and comprising the sequences disclosed in TABLE 1: (1) the 6 CDR amino acid sequences (CDRH1, CDRH2, CDRH3, CDRL1, CDRL2, CDRL3); (2) the heavy chain variable region (VH) and the light chain variable region (VL); (3) the a heavy chain and the light chain; or (4) two heavy chains and two light chains.

The invention also provides for olaratumab for use in the treatment of a gastrointestinal stromal tumor with a PDGFRα mutation wherein the PDGFRα mutation is D842V.

The invention also provides for olaratumab for use in the treatment of a gastrointestinal stromal tumor with a PDGFRα mutation wherein the olaratumab is administered at a dose of about 20 mg/kg.

The invention also provides for olaratumab for use in the treatment of a gastrointestinal stromal tumor with a PDGFRα mutation wherein the PDGFRα mutation is D842V and wherein the olaratumab is administered at a dose of about 20 mg/kg.

As used herein, the term “antigen” includes a protein located on a cell's surface. Antigens can include polypeptides, carbohydrates, nucleic acids, lipids, haptens or other naturally occurring or synthetic compounds. Preferably, the antigen is a folded polypeptide or protein. Specific ligands bind the protein or receptor, initiating signal transduction and a change in cellular activity. Antibodies can also bind the antigen which can block ligand binding and the resulting signal transduction. The terms antigen, “receptor,” “target” or “target antigen” are used interchangeably herein.

The terms “platelet-derived growth factor receptor alpha,” “platelet-derived growth factor receptor α,” “PDGFR alpha,” “PDGFRα,” “PDGF alpha receptor,” and “PDGFα receptor” are used interchangeably herein, unless otherwise indicated, and are intended to refer to the human type III receptor tyrosine kinase, as well as functionally active, mutated forms thereof, that bind human platelet-derived growth factor. Specific examples of PDGFRα include, e.g., a human polypeptide encoded by the nucleotide sequence provided in GenBank® accession no. NM_006206.4 (SEQ ID NO 13), or the human protein encoded by the polypeptide sequence provided in GenBank® accession no. NP_006197.1.

PDGFRα is a receptor tyrosine kinase that can be activated by platelet-derived growth factor (PDGF)-AA, -AB, -BB, and -CC. These growth factors are dimeric molecules composed of disulfide-linked polypeptide chains that bind to two receptors simultaneously and induce receptor dimerization, autophosphorylation, and down-stream intracellular signaling. PDGFRα is expressed in many mesenchymal structures and PDGFRα plays a critical role during early and later stages of development.

As used herein, the term “mutation” includes changes in the nucleotide sequence of the genome including changes in the amino acid sequence of the antigen. Mutations are an anomaly or change in the sequence of the antigen that deviates from what is wild type, standard, normal or expected.

Mutations in the receptor may be determined in a diagnostic or prognostic assay by evaluating extracted and purified DNA of the PDGFRα exons (specifically exons 12, 14, and 18) by direct, bidirectional sequencing, or real time PCR amplification followed by DNA sequencing. Other methods to detect the mutations include RNA sequencing, high resolution melting (HRM) techniques and nucleotide hybridization.

The presence of a mutation may be detected in a sample taken from the patient. The patient sample may be blood, serum, plasma, urine, tissue, tumor cells, tumor tissue samples, circulating tumor cells, and circulating DNA.

As used herein, the term “olaratumab”—also known as IMC-3G3, CAS registry number 1024603-93-7—refers to an anti-PDGFRα antibody comprising: two heavy chains, each of whose amino acid sequence is that given in SEQ ID NO: 9, and two light chains, each of whose amino acid sequence is that given in SEQ ID NO: 10. U.S. Pat. Nos. 8,128,929 and 8,574,578.

Olaratumab is a recombinant human monoclonal antibody of the IgG₁ isotype that specifically targets human PDGFRα. The antibody possesses high-affinity binding for PDGFRα and blocks platelet-derived growth factor-AA (PDGF-AA), -BB, and -CC ligands from binding to the receptor. As a result, olaratumab inhibits ligand-induced receptor autophosphorylation and phosphorylation of the downstream signaling molecules protein kinase B (Akt) and mitogen-activated protein kinase (MAPK). Olaratumab inhibits the proliferation and growth of a variety of human tumor cell lines.

As used herein, the term “antibody” includes immunoglobulin molecules comprising four polypeptide chains: two heavy (H) chains and two light (L) chains inter-connected by disulfide bonds. Individual chains can fold into domains having similar sizes (110-125 amino acids) and structures, but different functions. Antibody may be abbreviated herein as “Ab.”

The light chain can comprise one variable domain (VL) and/or one constant domain (abbreviated herein as CL). The light chains of human antibodies (immunoglobulins) are either kappa (κ) light chains or lambda (λ) light chains. The expression VL, as used herein, is intended to include both the variable regions from kappa-type light chains (VK) and from lambda-type light chains (Vλ). The heavy chain can also comprise one variable domain (VH) and/or, depending on the class or isotype of antibody, three or four constant domains (CH1, CH2, CH3 and CH4) (abbreviated herein collectively as CH). In humans, the isotypes are IgA, IgD, IgE, IgG, and IgM, with IgA and IgG further subdivided into subclasses or subtypes (IgA₁₋₂ and IgG₁₋₄). The present invention includes antibodies of any of the aforementioned classes or subclasses. Human IgG₁ is the preferred isotype for the antibodies of the present invention.

Three regions, called hypervariable or complementarity-determining regions (hereinafter “CDRs”), are found in each of VL and VH, which are supported by less variable regions called frameworks (herein as “FR”). Amino acids are assigned to a particular CDR region or domain in accordance with various conventions including, but not limited to: Kabat (Kabat, et al., Sequences of Proteins of Immunological Interest, Fifth Edition, U.S. Department of Health and Human Services, NIH Publication No. 91-3242 (1991)), Chothia (Chothia, et al., J Mol Biol. 1987; 196: 901-917. Chothia, et al., Nature. 1989; 342: 877-883), and/or Oxford Molecular AbM antibody modelling software (http://www.bioinf.org.uk/abs/). Each VH and VL is composed of three CDRs and four FRs, arranged from amino-terminus to carboxy-terminus in the following order: FR1-CDR1-FR2-CDR2-FR3-CDR3-FR4. The portion of an antibody consisting of VL and VH domains is designated Fv (Fragment variable) and constitutes the antigen-binding site.

The term “isolated” refers to an antibody, protein, peptide or nucleic acid that is free or substantially free from other macromolecular species found in a cellular environment. “Substantially free,” as used herein means the protein peptide or nucleic acid of interest comprises more than 80% (on a molar basis) of the macromolecular species present, preferably more than 90% and more preferably more than 95%. Examples of “isolated” antibodies include an antibody that has been affinity purified, an antibody that has been made by a hybridoma or other cell line in vitro, and a human antibody derived from a transgenic mouse.

The term “monoclonal,” as used herein, refers to an antibody obtained from a population of substantially homogeneous antibodies, e.g., the individual antibodies comprising the population are substantially identical except for possible naturally occurring mutations or minor post-translational variations that may be present. Monoclonal antibodies are highly specific, being directed against a single antigenic site (also known as determinant or epitope). Furthermore, in contrast to conventional (polyclonal) antibody preparations that typically include different antibodies directed against different determinants, each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibody may be abbreviated herein as “mAb.”

The term “human antibody,” as used herein, includes antibodies having variable and constant regions corresponding to human germline immunoglobulin sequences (as described in Kabat et al., supra). The human antibodies of the invention may include amino acid residues not encoded by human germline immunoglobulin sequences (e.g., mutations introduced by random or site-specific mutagenesis in vitro or by somatic mutation in vivo), for example in the CDRs. The human antibody can have at least one position replaced with an amino acid residue, e.g., an activity enhancing amino acid residue which is not encoded by the human germline immunoglobulin sequence. However, the term “human antibody,” as used herein, is not intended to include antibodies in which CDR sequences derived from the germline of another mammalian species, such as a mouse, have been grafted onto human framework sequences. Methods of producing a “human antibody,” as used herein are not intended to include antibodies produced in a human being.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal that is transgenic for human immunoglobulin genes, or antibodies prepared, expressed, created or isolated by any other means that involves splicing of human immunoglobulin gene sequences to other DNA sequences. Such recombinant human antibodies have variable and constant regions derived from human germline immunoglobulin sequences.

Thus, antibodies of the invention include, but are not limited to, isolated antibodies, human antibodies, humanized antibodies, recombinant human antibodies, monoclonal antibodies, digestion fragments, specified portions and variants thereof, including antibody mimetics or comprising portions of antibodies that mimic the structure and/or function of an antibody or specified fragment or portion thereof; each containing at least one CDR.

Specificity of antibodies or fragments thereof can be determined based on affinity. Affinity, represented by the equilibrium constant for the dissociation of an antigen with an antibody (K_{D}), measures the binding strength between an antigenic determinant and an antibody-binding site. Affinity can be measured for example by surface plasmon resonance.

The antibodies of the invention bind to an epitope of PDGFRα located on the extracellular domain segments (hereinafter referred simply to as “domains” or “ECD”). The term “epitope” as used herein refers to discrete, three-dimensional sites on an antigen that are recognized by the antibodies of the invention.

In addition to the antibodies specifically described herein, other “substantially homologous” modified antibodies can be readily designed and manufactured utilizing various recombinant DNA techniques well known to those skilled in the art. For example, the framework regions can vary from the native sequences at the primary structure level by several amino acid substitutions, terminal and intermediate additions and deletions, and the like. Moreover, a variety of different human framework regions may be used singularly or in combination as a basis for the humanized immunoglobulins of the present invention. In general, modifications of the genes may be readily accomplished by a variety of well-known techniques, such as site-directed mutagenesis.

The present invention includes nucleic acid sequences that encode an anti-PDGFRα antibody heavy chain, comprising any one of the VH regions or a portion thereof, or any one of the VH CDRs, including any variants thereof, as disclosed herein. The invention also includes nucleic acid molecules that encode an anti-PDGFRα antibody light chain comprising any one of the VL regions, or a portion thereof or any one of the VL CDRs, including any variants thereof as disclosed herein. The invention also includes the nucleic acid sequences of olaratumab, SEQ ID NOs 11 and 12 for heavy chain and light chain, respectively. The antibodies of the invention include antibodies comprising the same CDR regions of olaratumab, and/or the same light chain variable region and/or heavy chain variable region of olaratumab.

The antibodies of the present invention may be produced by methods known in the art. These methods include the use of transgenic animal, phage display and the immunological method described by Kohler and Milstein, Nature 256: 495-497 (1975); Laboratory Techniques in Biochemistry and Molecular Biology, Volume 13 (Burdon et al. eds., Elsevier Science Publishers, Amsterdam) in Monoclonal Antibody Technology, The Production and Characterization of Rodent and Human Hybridomas (Campbell ed., 1984); as well as by the recombinant DNA method described by Huse et al., Science 246: 1275-1281 (1989).

It is understood that amino acid residues that are primary determinants of binding of single domain antibodies can be within Kabat, Chothia, AbM, or a combination thereof defined CDRs, but may include other residues as well, such as, for example, residues that would otherwise be buried in the VH-VL interface of a VH-VL heterodimer.

Preferred host cells for transformation of vectors and expression of the antibodies of the present invention are mammalian cells, e.g., NSO cells, 293, SP20 and CHO cells and other cell lines of lymphoid origin such as lymphoma, myeloma, or hybridoma cells. Other eukaryotic hosts, such as yeasts, can be alternatively used.

The antibodies of the present invention may be isolated or purified by any method known in the art, including precipitation by ammonium sulfate or sodium sulfate followed by dialysis against saline, ion exchange chromatography, affinity or immuno-affinity chromatography, as well as gel filtration or zone electrophoresis. A preferred method of purification for the antibodies of the current invention is Protein-A affinity chromatography.

As used herein, “about” means±5%.

As used herein, the terms “treating,” “treat,” or “treatment” refers to restraining, slowing, lessening, reducing, or reversing the progression or severity of an existing symptom, disorder, condition, or disease or ameliorating clinical symptoms of a condition. Beneficial or desired clinical results include, but are not limited to, alleviation of symptoms, diminishment of the extent of a disease or disorder, stabilization of a disease or disorder (i.e., where the disease or disorder does not worsen), delay or slowing of the progression of a disease or disorder, amelioration or palliation of the disease or disorder, and remission (whether partial or total) of the disease or disorder, whether detectable or undetectable. Treatment can also mean prolonging survival as compared to expected survival if not receiving treatment. Those in need of treatment include those already with the disease. In one embodiment, the present invention can be used as a medicament.

As used herein, the terms “cancer” and “cancerous” refer to or describe the physiological condition in mammals that is typically characterized by unregulated cell growth. Included in this definition are benign and malignant cancers.

Although human antibodies of the invention are particularly useful for administration to humans, they can be administered to other mammals as well. Accordingly, as used herein, the term “patient” refers to a mammal, preferably a human. The term mammal as used herein is intended to include, but is not limited to, humans, laboratory animals, domestic pets and farm animals.

In the methods of the present invention, a therapeutically effective amount of an antibody of the invention is administered to a mammal or patient in need thereof. Additionally, the pharmaceutical compositions of the invention may include a therapeutically effective amount of an anti-PDGFRα antibody of the invention.

A “therapeutically effective amount,” “effective amount” or “effective dose” as used herein, refers to an amount effective, at dosages and for periods of time necessary, to achieve the desired therapeutic result. An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the target site; the degree of or involvement or the severity of the disease or disorder; the response of the individual patient; the particular compound administered; the mode of administration; the bioavailability characteristics of the preparation administered; the dose regimen selected; the use of concomitant medication; other medications administered; and other relevant circumstances. A therapeutically effective amount is also one in which any toxic or detrimental effects of the antibody or antibody portion are outweighed by the therapeutically beneficial effects.

Generally, dosage regimens may be adjusted to provide the optimum desired response (e.g., a therapeutic response). Treatment dosages may be titrated using routine methods known to those of skill in the art to optimize safety and efficacy. Dosing schedules will typically range from a single bolus dosage or continuous infusion to multiple administrations per day (e.g., every 4-6 hours), or as indicated by the treating physician and the patient's condition. An exemplary, non-limiting range for a therapeutically effective amount of an antibody of the invention is 0.1-50 mg/kg, more preferably 3-35 mg/kg, and more preferably 5-20 mg/kg. Dosing amounts and frequencies of the antibody will be determined by the physicians treating the patient and may include doses from less than 1 mg/kg to over 100 mg/kg given daily, three times per week, weekly, once every two weeks, or less often. It should be noted, however, that the present invention is not limited to any particular dose.

Olaratumab is generally effective over a wide dosage range in the present invention. For example, dosages normally are given on a 14-day cycle and each dose falls within the range of about 10 mg/kg to about 30 mg/kg, preferably about 18.5 mg/kg to about 22.5 mg/kg, and most preferably about 20 mg/kg. In one aspect of the invention, patients may be treated in cycles of 14 days until evidence of confirmed disease progression.

In some instances, dosage levels below the lower limit of the aforesaid ranges for olaratumab may be more than adequate, while in other cases smaller or still larger doses may be employed with acceptable side effects, and therefore the above dosage range is not intended to limit the scope of the invention in any way.

As used herein, the terms “effective response” of a patient or a patient's “responsiveness” to treatment with of the agents, or “therapeutic effect” refers to the clinical or therapeutic benefit(s) imparted to a patient upon administration. As used herein, an “unexpected therapeutic effect” of the treatment of the invention is the ability to produce marked anti-cancer effects in a patient without causing significant toxicities or adverse effects, so that the patient benefits from the treatment overall. The efficacy, i.e., therapeutic effect(s), of the treatment of the invention can be measured by various endpoints commonly used in evaluating cancer treatments, include any one or more including, but not limited to: extending survival (including OS and PFS); resulting in an objective response (including a CR or a PR); tumor regression, tumor weight or size shrinkage, longer time to disease progression, increased duration of survival, longer PFS, improved OS rate, increased duration of response, and improved quality of life and/or improving signs or symptoms of cancer, etc.

As used herein, the term “progressive disease” (PD) refers to least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progression.

As used herein, the term “partial response,” (PR) refers to at least a 30% decrease in the sum of diameters of target lesions, taking as reference the baseline sum diameters.

As used herein, the term “complete response” (CR) refers to the disappearance of all non-nodal target lesions with the short axes of any target lymph nodes reduced to <10 mm.

As used herein, the term “stable disease” (SD) refers to neither sufficient shrinkage for PR or sufficient increase to qualify for PD, taking as reference the smallest sum diameters while on Study.

As used herein, the term “objective response rate” (ORR) is equal to the proportion of patients achieving a best overall response of partial or complete response (PR+CR) according to RECIST 1.1.

As used herein, the term “overall survival” (OS) refers to the percentage of patients remaining alive for a defined period of time, such as 1 year, 5 years, etc. from the time of diagnosis or treatment. In a preferred aspect of the invention, for the Study, overall survival is defined as the time from the date of randomization in the Study to the date of death from any cause; if the patient is alive at the end of the follow-up period or is lost to follow-up, OS will be censored on the last date the patient is known to be alive.

As used herein, the term “progression-free survival” (PFS) refers to the patient remaining alive without the cancer progressing or getting worse. In a preferred aspect of the invention, PFS is defined as the time from randomization in the Study until the first radiographic documentation of objective progression as defined by RECIST (Version 1.1), or death from any cause. Patients who die without a reported prior progression will be considered to have progressed on the day of their death. Patients who did not progress or are lost to follow-up will be censored at the day of their last radiographic tumor assessment.

As used herein, the term “disease control rate” (DCR) refers to lack of disease progression and rate thereof. It refers to the group of patients with a best overall response categorized as CR, PR or SD (specifically excluding the patients with PD), wherein the best overall response is the best response recorded from the start of treatment until PD.

As used herein, the terms “clinical benefit rate,” refer to SD or better at 12 weeks. The tumor response rate of SD or better (i.e. CR+PR+SD) at 12 weeks is defined as the proportion of patients with a response of SD or better, as defined by RECIST 1.1, at 12 weeks following the first dose of study therapy. Patients will be considered “failure” if they die or if radiographic evaluation indicates a response of PD at 12 weeks or before.

As used herein, the term “extending survival” or “prolonged survival” which are used interchangeably herein, is meant as increasing OS or PFS in a treated patient relative to i) an untreated patient, ii) a patient treated with less than all of the anti-tumor agents in a particular combination therapy, or iii) a control treatment protocol. Survival is monitored following the initiation of treatment or following the initial diagnosis of cancer.

In the present invention, any suitable method or route can be used to administer anti-PDGFRα antibodies of the invention; intravenous (i.v.) administration is the preferred route. It should be emphasized, however, that the present invention is not limited to any particular method or route of administration.

The anti-PDGFRα antibodies of the invention, where used in a mammal for the purpose of treatment, are preferably formulated as pharmaceutical compositions. Such pharmaceutical compositions and processes for preparing the same are well known in the art. See, e.g. Remenigton: The Science and Practice of Pharmacy (Gennaro A., et al., eds., 19th ed., Mack Publishing Co., 1995).

Olaratumab is preferably formulated as pharmaceutical compositions administered by any route which makes the compound bioavailable. The route of administration may be varied in any way, limited by the physical properties of the drugs and the convenience of the patient and the caregiver. Preferably, olaratumab compositions are for parenteral administration, such as i.v. administration. Such pharmaceutical compositions and processes for preparing same are well known in the art. (See e.g., id.). The route of administration may be varied in any way, limited by the physical properties of the drugs and the convenience of the patient and the caregiver.

The following examples illustrate the unexpected benefit of the present invention.

### EXAMPLES AND ASSAYS

The following examples and assays further illustrate the invention, but should not be construed to limit the scope of the invention in any way. Detailed descriptions of conventional methods, such as those employed in the construction of vectors and plasmids, the insertion of genes encoding polypeptides into such vectors and plasmids, the introduction of plasmids into host cells, and the expression and determination thereof of genes and gene products can be obtained from numerous publications, including Sambrook, J. et al., Molecular Cloning: A Laboratory Manual, 2nd ed., Cold Spring Harbor Laboratory Press (1989) and Coligan, J. et al. Current Protocols in Immunology, Wiley & Sons, Incorporated (2007).

### Engineering, Expression and Purification of Human Anti-PDGFRα Antibodies

For each antibody (U.S. Pat. Nos. 8,128,929 and 8,574,578), engineer a suitable heavy chain nucleotide sequence, for example SEQ ID NO. 11 for olaratumab, into a suitable expression plasmid and engineer a suitable light chain nucleotide sequence, for example SEQ ID NO. 12 for olaratumab, into a suitable expression plasmid by a suitable method such as PCR cloning. To establish a stable cell line, transfect in a suitable host cell line, such as NSO or CHO cells, with linearized heavy and light chain plasmids and culture in suitable media such as glutamine free Dulbecco's Modified Eagle Medium with dialyzed fetal calf serum and glutamine synthetase supplement. Screen clones for antibody expression by an enzyme-linked immunosorbent assay (ELISA) and select the highest producer for culture in spinner flasks. Purify antibodies by a suitable method such as protein-A affinity chromatography.

Table 1 provides the amino acid sequences and corresponding SEQ ID NOs. of the antibody of the present invention. All CDR sequences are determined using the Kabat convention. Polynucleic acid sequences that encode the amino acid sequences disclosed below are also included within the scope of the present invention.

TABLE 1
Amino Acid Sequence of olaratumab
Heavy and Light Chain Variable Region CDRs

| | | SEQ | | SEQ |
|---|---|---|---|---|
| | | ID | | ID |
| | Heavy Chain | NO. | Light Chain | NO. |
| CDR1 | SSSYYWG | 1 | RASQSVSSYLA | 4 |
| CDR2 | SFFYTGSTYYNPSLRS | 2 | DASNRAT | 5 |
| CDR3 | QSTYYYGSGNYYGWFDR | 3 | QQRSNWPPA | 6 |
| Variable | QLQLQESGPGLVKPSETLSLT | 7 | EIVLTQSPATLSLSPGERA | 8 |
| region | CTVSGGSINSSSYYWGWLRQ | | TLSCRASQSVSSYLAWYQ | |
| | SPGKGLEWIGSFFYTGSTYY | | QKPGQAPRLLIYDASNRA | |
| | NPSLRSRLTISVDTSKNQFSL | | TGIPARFSGSGSGTDFTLTI | |
| | MLSSVTAADTAVYYCARQS | | SSLEPEDFAVYYCQQRSN | |
| | TYYYGSGNYYGWFDRWDQ | | WPPAFGQGTKVEIK | |
| | GTLVTVSS | | | |
| Full | MGWSCIILFLVATATGVHSQ | 9 | MGWSCIILFLVATATGVH | 10 |
| Length | LQLQESGPGLVKPSETLSLTC | | SEIVLTQSPATLSLSPGERA | |
| | TVSGGSINSSSYYWGWLRQS | | TLSCRASQSVSSYLAWYQ | |
| | PGKGLEWIGSFFYTGSTYYN | | QKPGQAPRLLIYDASNRA | |
| | PSLRSRLTISVDTSKNQFSLM | | TGIPARFSGSGSGTDFTLTI | |
| | LSSVTAADTAVYYCARQST | | SSLEPEDFAVYYCQQRSN | |
| | YYYGSGNYYGWFDRWDQG | | WPPAFGQGTKVEIKRTVA | |
| | TLVTVSSASTKGPSVFPLAPS | | APSVFIFPPSDEQLKSGTA | |
| | SKSTSGGTAALGCLVKDYFP | | SVVCLLNNFYPREAKVQ | |
| | EPVTVSWNSGALTSGVHTFP | | WKVDNALQSGNSQESVT | |
| | AVLQSSGLYSLSSVVTVPSSS | | EQDSKDSTYSLSSTLTLSK | |
| | LGTQTYICNVNHKPSNTKVD | | ADYEKHKVYACEVTHQG | |
| | KRVEPKSCDKTHTCPPCPAP | | LSSPVTKSFNRGEC | |
| | ELLGGPSVFLFPPKPKDTLMI | | | |
| | SRTPEVTCVVVDVSHEDPEV | | | |
| | KFNWYVDGVEVHNAKTKPR | | | |
| | EEQYNSTYRVVSVLTVLHQ | | | |
| | DWLNGKEYKCKVSNKALPA | | | |
| | PIEKTISKAKGQPREPQVYTL | | | |
| | PPSREEMTKNQVSLTCLVKG | | | |
| | FYPSDIAVEWESNGQPENNY | | | |
| | KTTPPVLDSDGSFFLYSKLTV | | | |
| | DKSRWQQGNVFSCSVMHEA | | | |
| | LHNHYTQKSLSLSPGK | | | |

### A Randomized Phase 2 Study Evaluating the Efficacy of Olaratumab in the Treatment of Unresectable and/or Metastatic GIST (“the Study”)

### Study Design

The Study is an open-label, multinational, multicenter, Phase 2 clinical trial evaluating the safety and efficacy of olaratumab in the treatment of unresectable and/or metastatic GIST. Patients in this study are considered eligible if they have histologically or cytologically confirmed, unresectable and/or metastatic GIST with objective progression following, or intolerance to, treatment with at least both imatinib and sunitinib.

Enrolled patients who meet all eligibility criteria are separated into two molecularly distinct cohorts: Cohort 1 includes patients with GIST harboring PDGFRα mutations (D842V and any others) (hereinafter “Mutated Cohort”) while Cohort 2 includes patients with GIST not harboring PDGFRα mutations (hereinafter “Wild-Type Cohort”).

All patients receive 20 mg/kg of olaratumab administered intravenously (i.v.) over 1 hour every 2 weeks (14 days, one cycle) in the absence of disease progression or other withdrawal criteria. The dose of olaratumab depends upon the patient's baseline body weight in kilograms. Actual body weight is used for dose calculation. Infusions administered within 3 days before or after the planned infusion time point are considered acceptable. Patients are assessed for tumor response every 6 weeks (±3 days). All patients are to continue to receive treatment until there is radiographic documentation of disease progression, death, or intolerable toxicity, or other withdrawal criteria are not met.

### Efficacy Analysis

Efficacy outcomes are assessed by imaging studies and tumor measurements/disease response assessments according to RECIST 1.1 every 6 weeks (±3 days) after the first dose of study therapy until documentation of PD.

Clinical benefit rate at 12 weeks (primary efficacy endpoint) and PFS, OS, ORR, and DCR (secondary efficacy endpoints) are analyzed statistically. Clinical benefit rate at 12 weeks and its 90% binomial exact confidence limit are estimated for each cohort. The Kaplan-Meier method is used to estimate the median PFS time and PFS rate at 12 weeks for each cohort, together with their 90% confidence intervals (CIs). Overall survival is estimated by the Kaplan-Meier method for each cohort, and a 90% CI is provided for the median OS. The ORR is equal to the proportion of patients achieving a best overall response of PR or CR according to RECIST 1.1. For ORR, the number of patients achieving a response is divided by the total number of patients treated to yield the proportion responding. The ORR and 90% CI are also provided for each cohort. The number of patients achieving disease control is divided by the total number of patients treated to yield the DCR. The DCR and 90% CI are also provided for each cohort.

The first stage stopping rule for efficacy is based on the Evaluable Population (i.e., all eligible patients who receive at least 1 dose of study drug and undergo adequate tumor assessment at 12 weeks, including any patients discontinuing early due to PD or death). The primary efficacy endpoint is also analyzed for all patients included in the modified intent-to-treat (mITT) Population (i.e., all patients who receive any quantity of study drug). All other efficacy analyses are based on the mITT Population.

### Safety Analysis

Safety outcomes include adverse events (AEs), physical examinations, vital signs, electrocardiograms (ECGs), and clinical/laboratory tests. Safety analyses are based on the Safety Population (i.e., all patients who received any quantity of study drug).

### Results

A total of 30 patients were enrolled. Of these, 8 patients were considered screen failures and did not receive olaratumab, and 1 patient died of an AE before being assigned to a treatment. The remaining 21 patients were assigned to treatment and received at least 1 dose of olaratumab (Mutated Cohort, N=7; Wild-Type Cohort, N=14). All 21 patients were discontinued from the study for reasons including radiographically documented PD (18 patients, 85.7%), symptomatic deterioration of PD (2 patients, 9.5%), and death (1 patient, 4.8%). No patients discontinued from the study treatment due to AEs. All 21 patients were included in the mITT and Safety populations. Twenty patients were included in the Evaluable Population (one patient in the Mutated Cohort withdrew prior to 12 weeks and did not have a PD).

### Primary Efficacy

Primary efficacy analysis of Mutated Cohort (n=6) and Wild-Type Cohort (n=14), showed no CR or PR in the Evaluable population. Three patients (50.0%) in the Mutated Cohort and 2 patients (14.3%) in the Wild-Type Cohort had SD at 12 weeks. PD was observed in 3 patients (50.0%) in the Mutated Cohort and 12 patients (85.7%) in the Wild-Type Cohort. The clinical benefit rate at 12 weeks was 50.0% (90% CI, 15.3-84.7%) in the Mutated Cohort and 14.3% (90% CI, 2.6-38.5%) in the Wild-Type Cohort. See Table 2.

Similar to the Evaluable Population, there was no CR or PR in the mITT Population. Three (3) patients (42.9%) in the Mutated Cohort and two (2) patients (14.3%) in the Wild-Type Cohort had SD. PD was observed in three (3) patients (42.9%) in the Mutated Cohort and in 12 patients (85.7%) in the Wild-Type Cohort. The Clinical Benefit Rate was 42.9% (90% CI: 12.9-77.5%) in the Mutated Cohort and 14.3% (90% CI: 2.6-38.5%) in the Wild-Type Cohort.

TABLE 2
Tumor Response and Clinical Benefit Rate at 12 Weeks
(Evaluable Population)

| | |
|---|---|
| PDGFRα Mutated | PDGFRα Wild-Type |
| Cohort | Cohort |
| (n = 6) | (n = 14) |

Tumor Response at 12 weeks, n
(%)

| | | |
|---|---|---|
| SD | 3 (50.0) | 2 (14.3) |
| PD | 3 (50.0) | 12 (85.7) |
| Not evaluable | 0 | 0 |

Clinical Benefit Rate

| | | |
|---|---|---|
| n (%) | 3 (50.0) | 2 (14.3) |
| 90% CI | 15.3, 84.7 | 2.6, 38.5 |

### Secondary Efficacy

All secondary efficacy analyses were based on the mITT Population, consisting of all patients who received any quantity of study drug.

PFS:

As estimated by the Kaplan-Meier method, median PFS was 32.1 weeks (90% CI, 5.0-35.9 weeks) in the Mutated Cohort and 6.1 weeks (90% CI, 5.7-6.3 weeks) in the Wild-Type Cohort. In the Mutated Cohort, the 12- and 24-week PFS rates were both 51.4% (90% CI, 17.0-77.9%). In the Wild-Type Cohort, the 12- and 24-week PFS rates were 14.3% (90% CI, 3.4-32.7%) and not evaluable, respectively. See Table 3.

TABLE 3
PFS
(mITT Population)

| | PDGFRα Mutated | PDGFRα Wild-Type |
|---|---|---|
| | Cohort | Cohort |
| | (n = 7) | (n = 14) |
| Median, weeks | 32.1 | 6.1 |
| (90% CI) | (5.0-35.9) | (5.7-6.3) |
| 12-week PFS rate, % | 51.4 | 14.3 |
| (90% CI) | (17.0-77.9) | (3.4-32.7) |
| 24-week PFS rate, % | 51.4 | NE |
| (90% CI) | (17.0-77.9) | |

Abbreviations:
NE = not evaluable due to all patients having disease progression

OS:

In the Mutated Cohort, median OS was not reached, and the 6-month survival rate was 71.4% (90% CI, 33.9-90.1%). In the Wild-Type Cohort, median OS was 24.9 weeks (90% CI, 14.4-49.1 weeks) and the 6-month survival rate was 50.0% (90% CI, 27.1-69.2%).

ORR:

No CR or PR was observed in either cohort. Based on the mITT Population, 5 patients (71.4%) in the Mutated Cohort and 4 patients (28.6%) in the Wild-Type Cohort had SD. PD was observed in 2 patients (28.6%) in the Mutated Cohort and 10 patients (71.4%) in the Wild-Type Cohort.

Patients without PD:

Determination of percentages of patients that were not identified as having PD were based on patient progression data in the mITT Population which consists of all patients who received any quantity of study drug. See Table 4.

At ˜18 weeks, 43% of patients (3 of 7 patients) in the Mutated Cohort were identified as being without PD and 7% of patients (1 of 14 patients) in the Wild-Type Cohort were identified as being without PD.

At 32 weeks, 29% of patients (2 of 7 patients) in the Mutated Cohort were identified as being without PD and 0% of patients (0 of 14 patients) in the Wild-Type Cohort were identified as being without PD.

At 35 weeks, 14% of patients (1 of 7 patients) in the Mutated Cohort were identified as being without PD and 0% of patients (0 of 14 patients) in the Wild-Type Cohort were identified as being without PD.

TABLE 4
Patients Without PD
(mITT Population)

| | PDGFRα Mutated | PDGFRα Wild-Type |
|---|---|---|
| | Cohort | Cohort |
| ~18 weeks | 43% | 7% |
| 32 weeks | 29% | 0% |
| 35 weeks | 14% | 0% |

### Safety:

Finally, the toxicity profile of olaratumab is overall acceptable and well tolerated as compared to other GIST treatments with regard to adverse events. No distinct AE specifically indicating an olaratumab-related emerging trend could be identified.

The analysis of the clinical data from the Study illustrate 32.1 weeks of median PFS, which is a 26-week improvement in the PFS of patients with a PDGFRα mutation as compared to the PFS of PDGFRα wild-type or non-PDGFRα mutated patients (see Table 3). This 26-week improvement is a five-fold increase of the median PFS.

Furthermore, the median PFS in the Study for the PDGFRα mutated cohort of 32.1 weeks is a significant improvement over the 12.2-week median PFS for PDGFRα D842V mutated patients treated with imatinib as reported in Biron T. et. al. J Clin Oncol. 2010; 28:15s (suppl; abstr 10051).

Additionally, a clear trend can be seen in the length of time before patients with a PDGFRα mutation develop PD as compared to the length of time before PDGFRα wild-type or PDGFRα non-mutation patients develop PD (see Table 4). At 32 weeks, 29% of patients with a PDGFRα mutation were still showing a clinical benefit while none of the PDGFRα wild-type or non-PDGFRα mutated patients demonstrated a clinical benefit. Therefore, patients with a PDGFRα mutation treated with olaratumab had the clinical benefit of a prolonged time without PD as compared to wild-type patients when treated with olaratumab. This is an unexpected benefit from a clinical perspective.

As demonstrated herein, the outcomes in the PDGFRα-mutant and PDGFRα wild-type cohorts differ strikingly. Despite the small sample numbers, such a difference is unlikely to have been observed by chance. The disease stabilization observed in patients with progressive disease at study entry harboring PDGFRα-mutant GIST, a highly refractory population having no standard therapeutic options, is remarkable.

Finally, the toxicity profile of olaratumab is overall acceptable and well tolerated with regard to adverse events which are a critical, yet potentially elusive attribute for effective therapies.

### Additional Sequences

SEQ ID NO. 11
atgggatggtcatgtatcatcctttttctagtagcaactgcaactggagt
acattcacagctgcagctgcaggagtcgggcccaggactggtgaagcctt
cggagaccctgtccctcacctgcactgtctctggtggctccatcaacagt
agtagttactactggggctggctccgccagtccccagggaaggggctgga
gtggattgggagtttcttttatactgggagcacctactacaacccgtccc
tcaggagtcgactcaccatatccgtagacacgtccaagaaccagttctcc
ctgatgctgagttctgtgaccgccgcagacacggctgtatattactgtgc
gagacagtccacgtattactatggttcggggaattattatggctggttcg
accgctgggaccagggaaccctggtcaccgtctcctcagctagcaccaag
ggcccatcggtcttccccctggcaccctcctccaagagcacctctggggg
cacagcggccctgggctgcctggtcaaggactacttccccgaaccggtga
cggtgtcgtggaactcaggcgccctgaccagcggcgtgcacaccttcccg
gctgtcctacagtcctcaggactctactccctcagcagcgtggtgaccgt
gccctccagcagcttgggcacccagacctacatctgcaacgtgaatcaca
agcccagcaacaccaaggtggacaagagagttgagcccaaatcttgtgac
aaaactcacacatgcccaccgtgcccagcacctgaactcctggggggacc
gtcagtcttcctcttccccccaaaacccaaggacaccctcatgatctccc
ggacccctgaggtcacatgcgtggtggtggacgtgagccacgaagaccct
gaggtcaagttcaactggtacgtggacggcgtggaggtgcataatgccaa
gacaaagccgcgggaggagcagtacaacagcacgtaccgtgtggtcagcg
tcctcaccgtcctgcaccaggactggctgaatggcaaggagtacaagtgc
aaggtctccaacaaagccctcccagcccccatcgagaaaaccatctccaa
agccaaagggcagccccgagaaccacaggtgtacaccctgcccccatccc
gggaggagatgaccaagaaccaggtcagcctgacctgcctggtcaaaggc
ttctatcccagcgacatcgccgtggagtgggagagcaatgggcagccgga
gaacaactacaagaccacgcctcccgtgctggactccgacggctccttct
tcctctatagcaagctcaccgtggacaagagcaggtggcagcaggggaac
gtcttctcatgctccgtgatgcatgaggctctgcacaaccactacacgca
gaagagcctctccctgtccccgggtaaatga
SEQ ID NO. 12
atgggatggtcatgtatcatcctttttctagtagcaactgcaactggagt
acattcagaaattgtgttgacacagtctccagccaccctgtctttgtctc
caggggaaagagccaccctctcctgcagggccagtcagagtgttagcagc
tacttagcctggtaccaacagaaacctggccaggctcccaggctcctcat
ctatgatgcatccaacagggccactggcatcccagccaggttcagtggca
gtgggtctgggacagacttcactctcaccatcagcagcctagagcctgaa
gattttgcagtttattactgtcagcagcgtagcaactggcctccggcgtt
cggccaagggaccaaggtggaaatcaaacgtacggtggctgcaccatctg
tcttcatcttcccgccatctgatgagcagttgaaatctggaactgcctct
gttgtgtgcctgctgaataacttctatcccagagaggccaaagtacagtg
gaaggtggataacgccctccaatcgggtaactcccaggagagtgtcacag
agcaggacagcaaggacagcacctacagcctcagcagcaccctgacgctg
agcaaagcagactacgagaaacacaaagtctacgcctgcgaagtcaccca
tcagggcctgagctcgcccgtcacaaagagcttcaacaggggagagtgtt
ag
SEQ ID NO. 13
MGTSHPAFLVLGCLLTGLSLILCQLSLPSILPNENEKVVQLNSSFSLRCF
GESEVSWQYPMSEEESSDVEIRNEENNSGLFVTVLEVSSASAAHTGLYTC
YYNHTQTEENELEGRHIYIYVPDPDVAFVPLGMTDYLVIVEDDDSAIIPC
RTTDPETPVTLHNSEGVVPASYDSRQGFNGTFTVGPYICEATVKGKKFQT
IPFNVYALKATSELDLEMEALKTVYKSGETIVVTCAVFNNEVVDLQWTYP
GEVKGKGITMLEEIKVPSIKLVYTLTVPEATVKDSGDYECAARQATREVK
EMKKVTISVHEKGFIEIKPTFSQLEAVNLHEVKHFVVEVRAYPPPRISWL
KNNLTLIENLTEITTDVEKIQEIRYRSKLKLIRAKEEDSGHYTIVAQNED
AVKSYTFELLTQVPSSILDLVDDHHGSTGGQTVRCTAEGTPLPDIEWMIC
KDIKKCNNETSWTILANNVSNIITEIHSRDRSTVEGRVTFAKVEETIAVR
CLAKNLLGAENRELKLVAPTLRSELTVAAAVLVLLVIVIISLIVLVVIWK
QKPRYEIRWRVIESISPDGHEYIYVDPMQLPYDSRWEFPRDGLVLGRVLG
SGAFGKVVEGTAYGLSRSQPVMKVAVKMLKPTARSSEKQALMSELKIMTH
LGPHLNIVNLLGACTKSGPIYIITEYCFYGDLVNYLHKNRDSFLSHHPEK
PKKELDIFGLNPADESTRSYVILSFENNGDYMDMKQADTTQYVPMLERKE
VSKYSDIQRSLYDRPASYKKKSMLDSEVKNLLSDDNSEGLTLLDLLSFTY
QVARGMEFLASKNCVHRDLAARNVLLAQGKIVKICDFGLARDIMHDSNYV
SKGSTFLPVKWMAPESIFDNLYTTLSDVWSYGILLWEIFSLGGTPYPGMM
VDSTFYNKIKSGYRMAKPDHATSEVYEIMVKCWNSEPEKRPSFYHLSEIV
ENLLPGQYKKSYEKIHLDFLKSDHPAVARMRVDSDNAYIGVTYKNEEDKL
KDWEGGLDEQRLSADSGYIIPLPDIDPVPEEEDLGKRNRHSSQTSEESAI
ETGSSSSTFIKREDETIEDIDMMDDIGIDSSDLVEDSFL

1-25. (canceled)

26. A method of treating a gastrointestinal stromal tumor in a patient, comprising administering a therapeutically effective amount of olaratumab to the patient in need thereof, provided that a sample taken from the patient contains a PDGFR alpha mutation.

27. The method according claim 26, wherein the olaratumab is administered at a dose of about 20 mg/kg.

28. The method according to claim 26, wherein the PDGFR alpha mutation is D842V.

29. The method according to claim 26, wherein the sample is selected from the group consisting of blood, serum, plasma, urine, tissue, tumor cells, tumor tissue samples, circulating tumor cells, and circulating DNA.

30. A method of treating a gastrointestinal stromal tumor in a patient, comprising administering a therapeutically effective amount of olaratumab to the patient in need thereof, provided that the patient is selected for treatment on the basis of a sample taken from the patient that contains a PDGFR alpha mutation.

31. The method according claim 30, wherein the olaratumab is administered at a dose of about 20 mg/kg.

32. The method according to claim 30, wherein the PDGFR alpha mutation is D842V.

33. The method according to claim 30, wherein the sample is selected from the group consisting of blood, serum, plasma, urine, tissue, tumor cells, tumor tissue samples, circulating tumor cells, and circulating DNA.

34. A method of identifying a gastrointestinal stromal tumor patient eligible for treatment with olaratumab, comprising assaying for the presence of a PDGFR alpha mutation by DNA or RNA sequencing of a sample taken from the patient prior to the administration of a therapeutically effective amount of olaratumab, wherein the patient is eligible for treatment with olaratumab if the PDGFR alpha mutation is present in the sample.

35. The method according to claim 34, wherein the olaratumab is administered at a dose of about 20 mg/kg.

36. The method according to claim 34, wherein the PDGFR alpha mutation is D842V.

37. The method according to claim 34, wherein the sample is selected from the group consisting of blood, serum, plasma, urine, tissue, tumor cells, tumor tissue samples, circulating tumor cells, and circulating DNA.

38. A therapeutic regimen for treating a gastrointestinal stromal tumor, comprising: (1) selecting a patient having a gastrointestinal stromal tumor on the basis of a sample taken from the patient having a PDGFR alpha mutation, wherein the sample is selected from the group consisting of blood, serum, plasma, urine, tissue, tumor cells, tumor tissue samples, circulating tumor cells, and circulating DNA, and (2) administering to the patient olaratumab if the mutation is present.

39. The therapeutic regimen according to claim 38, wherein the mutation of PDGFR alpha is D842V.

40. The therapeutic regimen according to claim 38, wherein the olaratumab is administered at a dose of about 20 mg/kg.
